(19) United States
(12) Patent Application Publication
(10) Pub. No.: US 2013/0066320 A1
(43) Pub. Date: Mar. 14, 2013

(54) Medical Device

(71) Applicants: Marcus Jarman-Smith, Lytham St. Annes (GB); Gordon McDonough, Bolton (GB); Nuno Sereno, Southport (GB); Tony Whitehead, Poulton-le-Fylde (GB)

(72) Inventors: Marcus Jarman-Smith, Lytham St. Annes (GB); Gordon McDonough, Bolton (GB); Nuno Sereno, Southport (GB); Tony Whitehead, Poulton-le-Fylde (GB)

(73) Assignee: INVIBIO LIMITED, Thornton Cleveleys, Lancashire (GB)

(21) Appl. No.: 13/637,345

(22) PCT Filed: Mar. 23, 2011
(86) PCT No.: PCT/GB2011/050580

§ 371 Date: Nov. 28, 2012

(30) Foreign Application Priority Data
Mar. 26, 2010 (GB) 1005122.5

(51) Int. Cl. A61B 17/72; A61B 17/88

(52) U.S. Cl. CPC A61B 17/72; A61B 17/88
USPC 606/67; 156/322

(57) ABSTRACT

A titanium body (2) of a femoral nail incorporates an opening (3) leading into a hollow region (4) which defines a cavity in which electronics may be incorporated. A potting compound overlays the electronics, within the region (4). A 100 to 150 μm thick cover (18) made from polyetheretherketone is arranged to overlay the opening (3) and close the hollow region (4). The cover (18) is made from flat polyetheretherketone film of suitable thickness which is heat shrunk onto a former and then machined to define the desired shape.

This invention relates to a medical device and particularly, although not exclusively, relates to a method of closing an opening in a precursor of a medical device and a medical device formed thereby. Preferred embodiments, relate to medical devices in the form of nails, for example femoral nails.

Implantable devices are increasing in function and complexity. They may incorporate sensory loops between electrodes monitoring body/therapy behaviour and the communication of recorded data and control signals between the device and external systems. These signals are transmitted for example by means of radio frequency (RF) coupling. Moreover, devices are being developed which include batteries which are rechargeable by, for example, inductive coupling of power to a receiver coil.

The transmission of RF signals through an hermetic enclosure of an active implantable medical device may be affected by several factors, such as: a) the accuracy of the placement of the charging coil, b) the signal frequency, c) Eddy current losses in the housing, d) the charge rate of the battery and e) the Coulombic efficiency of the battery.

Current implantable devices are primarily enclosed in titanium alloy enclosures. However, a titanium alloy enclosure severely attenuates RF signals and generates increases in temperature due mainly to Eddy current losses associated with titanium material properties. Such effects mean that low operating frequencies have to be used and the battery recharging rate is decreased which has a detrimental effect on battery life.

Known methods of closing an opening in a medical device include the use of machined covers. However, such covers may be expensive to produce, particularly in low numbers and it may be very difficult to provide such covers with very thin (e.g. less than 200 μm) walls.

It is an object of the present invention to address the abovedescribed problems.

According to a first aspect of the invention, there is provided a method of making a medical device, the method comprising the steps of:

(a) selecting a film or tubing material which comprises a thermoplastics polymeric material;

(b) selecting a former;

(c) heat processing the film or tubing on the former so that the film or tubing adopts the shape of the former at least in part, thereby to prepare a cover;

(d) optionally treating the cover prepared in step (c) to adjust its shape;

(e) securing the cover over an opening in a precursor of the medical device in order to close the opening.

The medical device is preferably an implantable medical device. The device may have a maximum dimension (e.g. length) of at least 5 cm, 10 cm, 15 cm, 20 cm or 25 cm. The maximum dimension may be less than 50 cm or less than 40 cm. The maximum diameter of the device may be less than 5 cm, 4 cm, 3 cm or 2 cm. The diameter in at least a region of the device is suitably at least 0.5 cm.

The device is preferably elongate. It is preferably for stabilising fractures, for example femoral fractures. It is preferably a nail, for example a femoral nail.

The device preferably incorporates electronics for example, a battery, capacitor, microelectronic circuitry and/or an antenna. Such electronics are preferably arranged within the opening in the precursor.

The precursor of the medical device preferably comprises a metal, for example a biocompatible metal or metal alloy, with titanium being preferred. Walls defining the opening in the precursor preferably comprise and/or are defined by the metal or metal alloy, for example comprising titanium. Preferably, at least 60 wt %, 70 wt %, 80 wt %, 90 wt % or at least 95 wt % of the weight of the precursor (excluding any electronics) is made up of biocompatible metal, preferably titanium or an alloy thereof.

The opening in the precursor preferably includes a hollow region of the precursor in which electronics are arranged. The hollow region may include a potting material, for example a silicone, suitably to secure and/or protect the electronics with the device.

Said film or tubing material selected in step (a) may have a thickness in the range 25 μm to 1 mm. Suitably, the thickness is at least 50 μm, preferably at least 100 μm. The thickness may be less than 500 μm and is preferably in the range 100 to 300 μm. The method may more easily and cheaply allow relatively thin covers to be made and used, compared for example to covers made by alternative processes such as machining and/or injection moulding. Said film or tubing suitably has a substantially constant thickness across its extent.

Said film or tubing preferably comprises a polymeric material which has a moiety of formula

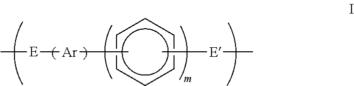

and/or a moiety of formula

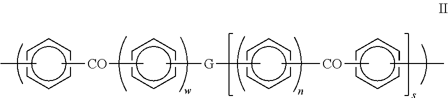

and/or a moiety of formula

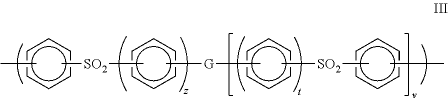

wherein m,r,s,t,v,w and z independently represent zero or a positive integer, E and E′ independently represent an oxygen or a sulphur atom or a direct link, G represents an oxygen or sulphur atom, a direct link or a —O-Ph-O— moiety where Ph represents a phenyl group and Ar is selected from one of the following moieties (i)**, (i) to (iv) which is bonded via one or more of its phenyl moieties to adjacent moieties

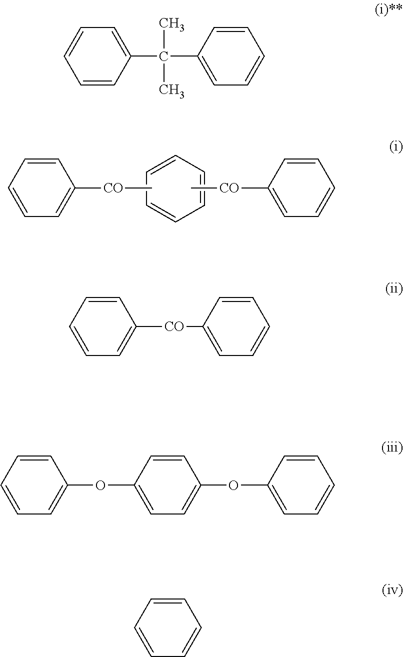

Unless otherwise stated in this specification, a phenyl moiety has 1,4-, linkages to moieties to which it is bonded.

In (i), the middle phenyl may be 1,4- or 1,3-substituted. It is preferably 1,4-substituted.

Said polymeric material may include more than one different type of repeat unit of formula I; and more than one different type of repeat unit of formula II; and more than one different type of repeat unit of formula III. Preferably, however, only one type of repeat unit of formula I, II and/or III is provided.

Said moieties I, II and III are suitably repeat units. In the polymeric material, units I, II and/or III are suitably bonded to one another—that is, with no other atoms or groups being bonded between units I, II and III.

Phenyl moieties in units I, II and III are preferably not substituted. Said phenyl moieties are preferably not cross-linked.

Where w and/or z is/are greater than zero, the respective phenylene moieties may independently have 1,4- or 1,3-linkages to the other moieties in the repeat units of formulae II and/or III. Preferably, said phenylene moieties have 1,4-linkages.

Preferably, the polymeric chain of the polymeric material does not include a —S— moiety. Preferably, G represents a direct link.

Suitably, “a” represents the mole % of units of formula I in said polymeric material, suitably wherein each unit I is the same; “b” represents the mole % of units of formula II in said polymeric material, suitably wherein each unit II is the same; and “c” represents the mole % of units of formula Ill in said polymeric material, suitably wherein each unit Ill is the same. Preferably, a is in the range 45-100, more preferably in the range 45-55, especially in the range 48-52. Preferably, the sum of b and c is in the range 0-55, more preferably in the range 45-55, especially in the range 48-52. Preferably, the ratio of a to the sum of b and c is in the range 0.9 to 1.1 and, more preferably, is about 1. Suitably, the sum of a, b and c is at least 90, preferably at least 95, more preferably at least 99, especially about 100. Preferably, said polymeric material consists essentially of moieties I, II and/or III.

Said polymeric material may be a homopolymer having a repeat unit of general formula

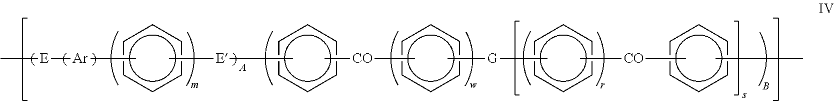

or a homopolymer having a repeat unit of general formula

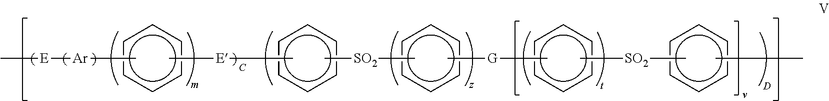

or a random or block copolymer of at least two different units of IV and/or V,wherein A, B, C and D independently represent 0 or 1 and E,E′,G,Ar,m,r,s,t,v,w and z are as described in any statement herein.

Preferably, m is in the range 0-3, more preferably 0-2, especially 0-1. Preferably, r is in the range 0-3, more preferably 0-2, especially 0-1. Preferably t is in the range 0-3, more preferably 0-2, especially 0-1. Preferably, s is 0 or 1. Preferably v is 0 or 1. Preferably, w is 0 or 1. Preferably z is 0 or 1.

Preferably, said polymeric material is a homopolymer having a repeat unit of general formula IV.

Preferably Ar is selected from the following moieties (xi)** and (vii) to (x)

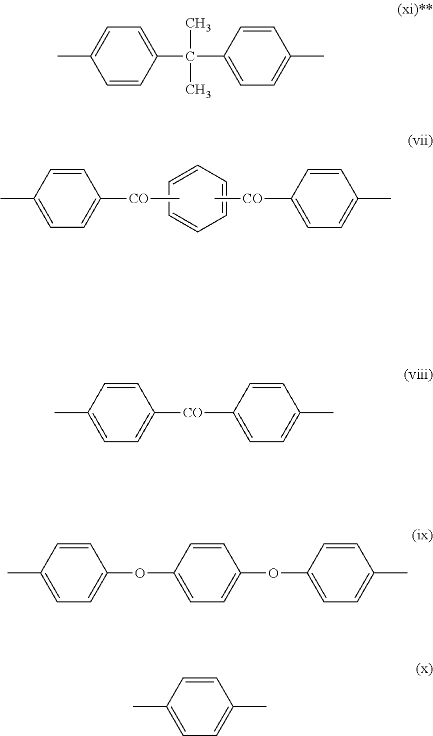

In (vii), the middle phenyl may be 1,4- or 1,3-substituted. It is preferably 1,4-substituted.

Suitable moieties Ar are moieties (i), (ii), (iii) and (iv) and, of these, moieties (i), (ii) and (iv) are preferred. Other preferred moieties Ar are moieties (vii), (viii), (ix) and (x) and, of these, moieties (vii), (viii) and (x) are especially preferred.

An especially preferred class of polymeric materials are polymers (or copolymers) which consist essentially of phenyl moieties in conjunction with ketone and/or ether moieties. That is, in the preferred class, said polymeric material does not include repeat units which include —S—, —SO₂— or aromatic groups other than phenyl. Preferred polymeric materials of the type described include:
(a) a polymeric material consisting essentially of units of formula IV wherein Ar represents moiety (iv), E and E′ represent oxygen atoms, m represents 0, w represents 1, G represents a direct link, s represents 0, and A and B represent 1 (i.e. polyetheretherketone).
(b) a polymeric material consisting essentially of units of formula IV wherein E represents an oxygen atom, E′ represents a direct link, Ar represents a moiety of structure (i), m represents 0, A represents 1, B represents 0 (i.e. polyetherketone);
(c) a polymeric material consisting essentially of units of formula IV wherein E represents an oxygen atom, Ar represents moiety (i), m represents 0, E′ represents a direct link, A represents 1, B represents 0, (i.e. polyetherketoneketone).
(d) a polymeric material consisting essentially of units of formula IV wherein Ar represents moiety (i), E and E′ represent oxygen atoms, G represents a direct link, m represents 0, w represents 1, r represents 0, s represents 1 and A and B represent 1. (i.e. polyetherketoneetherketoneketone).
(e) a polymeric material consisting essentially of units of formula IV, wherein Ar represents moiety (iv), E and E′ represents oxygen atoms, G represents a direct link, m represents 0, w represents 0, s, r, A and B represent 1 (i.e. polyetheretherketoneketone).
(f) a polymeric material comprising units of formula IV, wherein Ar represents moiety (iv), E and E′ represent oxygen atoms, m represents 1, w represents 1, A represents 1, B represents 1, r and s represent 0 and G represents a direct link (i.e. polyether-diphenyl-ether-phenyl-ketone-phenyl-).

Said polymeric material may be amorphous or semi-crystalline. Said polymeric material is preferably semi-crystalline. The level and extent of crystallinity in a polymer is preferably measured by wide angle X-ray diffraction (also referred to as Wide Angle X-ray Scattering or WAXS), for example as described by Blundell and Osborn (Polymer 24, 953, 1983). Alternatively, crystallinity may be assessed by Differential Scanning Calorimetry (DSC).

The level of crystallinity in said polymeric material may be at least 1%, suitably at least 3%, preferably at least 5% and more preferably at least 10%. In especially preferred embodiments, the crystallinity may be greater than 30%, more preferably greater than 40%, especially greater than 45%.

The main peak of the melting endotherm (Tm) for said polymeric material (if crystalline) may be at least 300° C.

Said polymeric material may consist essentially of one of units (a) to (f) defined above.

Said polymeric material preferably comprises, more preferably consists essentially of, a repeat unit of formula (XX)

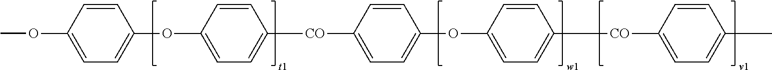

where t1, and w1 independently represent 0 or 1 and v1 represents 0, 1 or 2. Preferred polymeric materials have a said repeat unit wherein t1=1, v1=0 and w1=0; t1=0, v1=0 and w1=0; t1=0, w1=1, v1=2; or t1=0, v1=1 and w1=0. More preferred have t1=1, v1=0 and w1=0; or t1=0, v1=0 and w1=0. The most preferred has t1=1, v1=0 and w1=0.

In preferred embodiments, said polymeric material is selected from polyetheretherketone, polyetherketone, polyetherketoneetherketoneketone and polyetherketoneketone. In a more preferred embodiment, said polymeric material is selected from polyetherketone and polyetheretherketone. In an especially preferred embodiment, said polymeric material is polyetheretherketone.

Said polymeric material suitably has a melt viscosity (MV) of at least 0.06 kNsm⁻², preferably has a MV of at least 0.085 kNsm⁻², more preferably at least 0.12 kNsm⁻², especially at least 0.14 kNsm⁻².

MV is suitably measured using capillary rheometry operating at 400° C. at a shear rate of 1000 s⁻¹ using a tungsten carbide die, 0.5×3.175 mm.

Said polymeric material may have a MV of less than 1.00 kNsm⁻², preferably less than 0.5 kNsm⁻².

Said polymeric material may have a MV in the range 0.09 to 0.5 kNsm⁻², preferably in the range 0.14 to 0.5 kNsm⁻².

Said polymeric material may have a tensile strength, measured in accordance with ISO527 (specimen type 1b) tested at 23° C. at a rate of 50 mm/minute of at least 20 MPa, preferably at least 60 MPa, more preferably at least 80 MPa. The tensile strength is preferably in the range 80-110 MPa, more preferably in the range 80-100 MPa.

Said polymeric material may have a flexural strength, measured in accordance with ISO178 (80 mm×10 mm×4 mm specimen, tested in three-point-bend at 23° C. at a rate of 2 mm/minute) of at least 50 MPa, preferably at least 100 MPa, more preferably at least 145 MPa. The flexural strength is preferably in the range 145-180 MPa, more preferably in the range 145-164 MPa.

Said polymeric material may have a flexural modulus, measured in accordance with ISO178 (80 mm×10 mm×4 mm specimen, tested in three-point-bend at 23° C. at a rate of 2 mm/minute) of at least 1 GPa, suitably at least 2 GPa, preferably at least 3 GPa, more preferably at least 3.5 GPa. The flexural modulus is preferably in the range 3.5-4.5 GPa, more preferably in the range 3.5-4.1 GPa.

Said film or tubing preferably includes at least 60 wt %, 70 wt %, 80 wt %, 90 wt % or 95 wt % of said polymeric material. More preferably, said film or tubing consists essentially of said polymeric material. If said film or tubing includes material in addition to said polymeric material, it may include less than 10 wt %, preferably less than 5 wt % of other material, for example X-ray contrast material, such a barium sulphate.

Said former preferably has a shape which corresponds to the shape of at least a region surrounding the opening in the precursor of the medical device. Said former preferably includes a surface having a curved, for example arcuate cross-section. It may be made from a metal alloy (e.g. stainless steel), a polymeric material (e.g. PTFE), ceramics or a combination of the aforesaid. Suitably, the former has a relatively high melting temperature and is not deformable in use. The former may include a surface coating to facilitate release of the film or tubing.

In the method, the film/tubing and former are preferably contacted and subsequently heated to thermoform the polymeric material on the former.

In step (c), the film or tubing is suitably heated to deform it so that it adopts the shape of at least part of the former. Preferably, the film or tubing is heated to a temperature above the glass transition temperature (Tg) of the polymeric material of the film or tubing but at a temperature which is at least 5° C., preferably at least 10° C. less than the melting temperature (Tm) of the polymeric material. In one embodiment, a temperature of 330° to 360° may be applied for 5 minutes.

After step (c), the film/tubing is suitably allowed to cool. It may be removed from the former after cooling.

In optional step (d), the shape of the cover prepared in step (b) may be adjusted. For example, material may be removed from the cover, such as by machining or by manual trimming. Suitably, however, at this stage the cover is not deformed, for example by bending or thermoforming. Preferably, after step (c), there is no further deformation, for example bending or thermoforming, of the cover.

The cover may be treated prior to step (e) to facilitate its bonding to the precursor. Treatment may include grit blasting, chemical etching or treatment with plasma, corona, laser or UV light. Alternatively or additionally, a surface of the cover may be treated to effect physical/chemical modification to enhance or reduce cellular attachment and osteointegration. Such treatment may involve defining topography or involve functionalization for example by plasma or a bioactive coating.

Prior to step (e), a potting compound, for example a silicone, may be introduced to the opening. In step (e) the cover may contact and adhere to the potting compound which may facilitate securement of the cover in position.

In step (e), the cover prepared in step (c) and/or step (d) is suitably positioned over the opening suitably to completely close it (e.g. not to leave any gaps) and is secured in position. It may be secured in position by welding the cover to the precursor or by adhesive means. It is suitably secured to an area of biocompatible metal, for example comprising titanium, of the precursor of the medical device. Preferably, it is secured to an area of biocompatible metal which surrounds the opening in the precursor. It is preferably secured to an outwardly facing surface of the precursor. It is preferably secured to a curved surface (e.g. a convex) surface of the precursor.

Preferably, the cover is secured in position by adhesive means, suitably by adhesive contained, at least in part, in the opening.

Alternatively and/or additionally, the cover could be a press fit in the opening.

Once in position, the cover acts as a physical barrier over the opening which protects the electronics contained within the medical device whilst allowing transmission of signals to and/or from the electronics.

According to a second aspect of the invention, there is provided a medical device comprising a precursor of a medical device in which an opening is defined and a cover secured over the opening, said cover comprising a thermoplastics polymeric material. The medical device is suitably sterile.

The device may have any feature of the device of the first aspect. It suitably comprises a precursor in which an opening is defined, wherein suitably the precursor comprises a metal or metal alloy, for example comprising titanium. The cover preferably comprising or consists of a polymeric material of formula (XX) especially of polyetheretherketone. The cover may have a thickness, suitably across at least 60%, 70%, 80%, 90% or about 100% of a face which faces outwardly in use and/or is an exposed face of the device of 25 μm to 1 mm, preferably 50 μm to 500 μm, especially 100 μm to 300 μm. The cover preferably consists essentially of said face which is defined in an outer wall of the cover and opposed side walls which extend from the outer wall and extend inwardly in use.

According to a third aspect of the invention, there is provided a method of treating a human body, for example, repairing a fracture in a human body, the method using a medical device according to the second aspect.

The invention extends to the use of a medical device of the third aspect for treating a human body, for example repairing a fracture in a human body.

The invention extends to a package containing a medical device as described. The package is preferably sterile.

Any feature of any aspect of any invention or embodiment described herein may be combined with any feature of any aspect of any other invention or embodiment described herein mutatis mutandis.

Specific embodiments of the invention will now be described, by way of example, with reference to the accompanying drawings, in which:

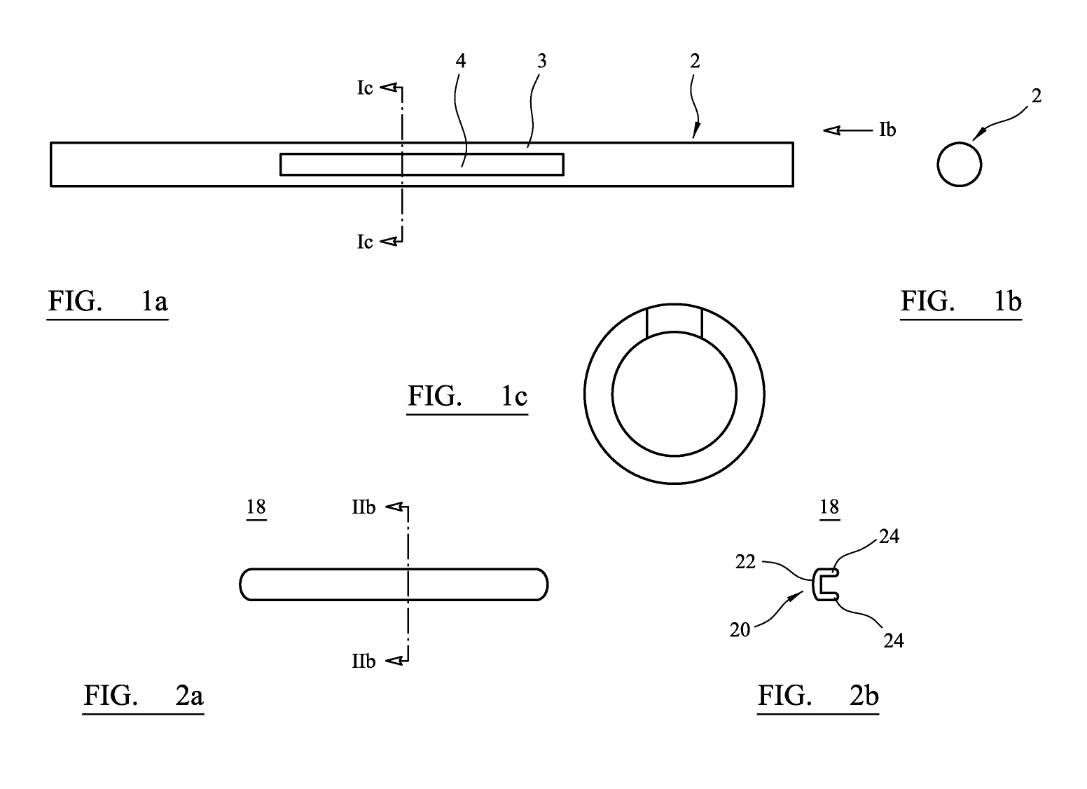

FIG. 1a is a plan view of a body of femoral nail;

FIG. 1b is an end view of the body in the direction of arrow Ib of FIG. 1a;

FIG. 1c is a cross-section along line Ic-Ic of FIG. 1a (on an enlarged scale);

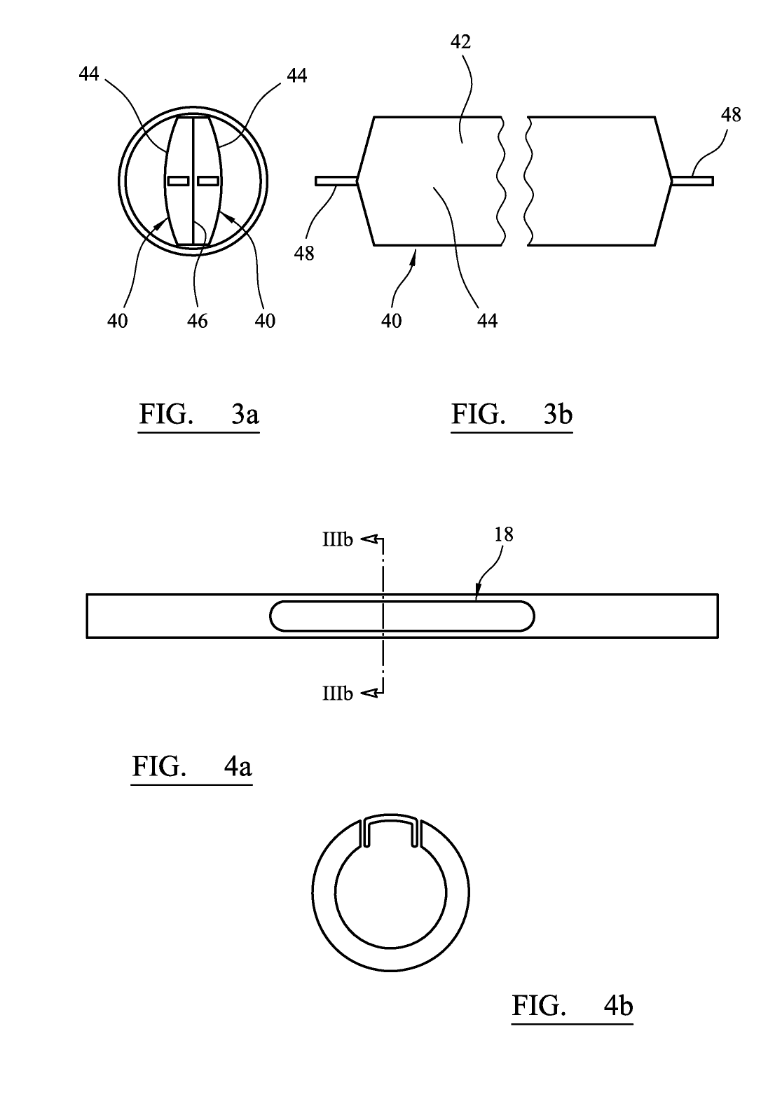

FIG. 2a is a plan view of a PEEK cover for the body;

FIG. 2b is a cross-section along line IIb-IIb of FIG. 2a (on an enlarged scale);

FIG. 3a is an end view of a PEEK tube with two formers inserted therein prior to heat shrinking;

FIG. 3b is a plan view of one of the formers of FIG. 3a;

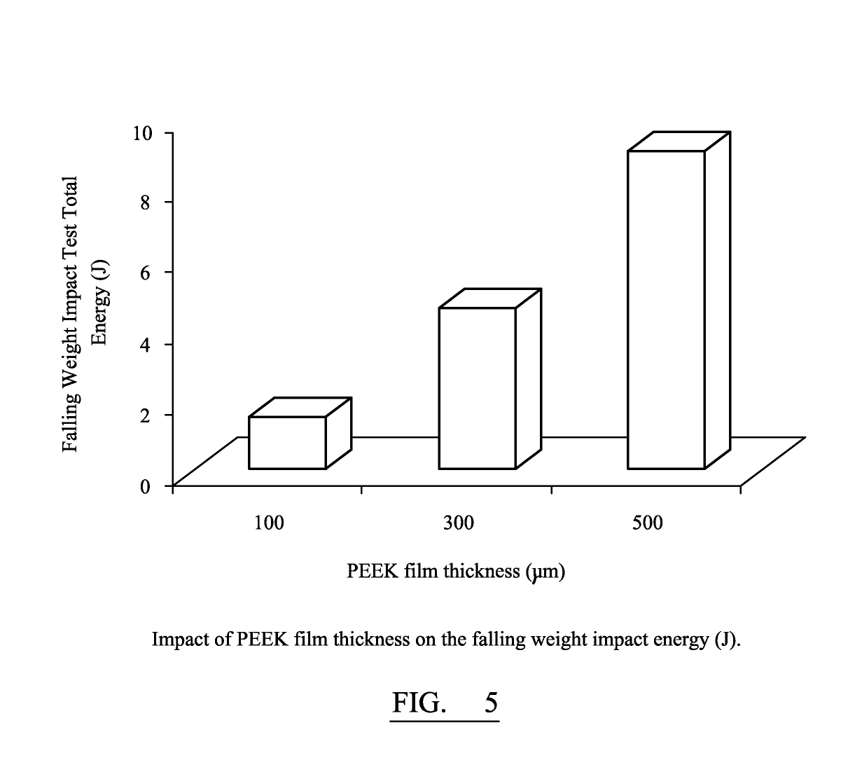

FIG. 4a is a plan view of a femoral nail comprising the body of FIG. 1 with the PEEK cover secured in position;

FIG. 4b is a cross-section along line IIIb-IIIb of FIG. 3a (on an enlarged scale); and

FIG. 5 is a plot of PEEK film thickness v. falling weight impact energy.

Referring to FIGS. 1a-1c, a body 2 of a femoral nail is made of metal alloys (e.g. titanium) along much of its extent except that it incorporates an opening 3 leading into a hollow region 4 which defines a cavity in which electronics (not shown) may be incorporated. The electronics are suitably arranged to allow communication between the nail, when implanted into a human body, and the outside A potting compound (not shown), for example comprising a silicone, suitably overlays the electronics and protects the electronics, to some extent, from water damage.

Referring to FIGS. 2a and 2b, a cover 18 made from polyetheretherketone (PEEK) is shown. It comprises a 100-150 μm thick shaped film of PEEK which is formed in a self-supporting shape (FIG. 2b) comprising an outer wall 20 having a convex outwardly facing surface 22 (having a radius of curvature generally corresponding to the radius of curvature of the nail surrounding opening 3) and spaced apart depending lateral portions 24.

The cover 18 may be made by selecting a flat PEEK film of suitable thickness, laying it on a former of suitable shape and then heat shrinking the film on the former so the film takes up the shape of at least part of the former. The film may be removed from the former and may be machined so it defines the desired shape.

Suitably, the former may be of the type illustrated in FIG. 3. Referring to FIG. 3a (which shows two formers 40) and FIG. 3b, a former 40, which may be made from metal, includes a main body 42 which has one curved surface 44 which has a radius of curvature which corresponds to the desired radius of curvature of outwardly facing surface 22 of the cover, and a flatter surface 46. Finger grip portions 48 are provided at each end of the body. In use, a piece of film is placed over the surface 44 of one former and heat shrunk thereby to define a precursor of the cover which may then be machined or otherwise treated to define the cover of FIG. 2b.

As an alternative to use of a film, a heat shrinkable tube made from PEEK may be selected and two formers 40 positioned therein as shown in FIG. 3a. The tube may then be heated so it shrinks around the two formers. Thereafter, the formers may be removed and the shrunken tube machined to define two substantially identical covers as shown in FIG. 2b.

A cover 18 may then be secured over the opening 3 to completely cover the opening and may be secured in position by suitable means. In one embodiment, it may be secured through the use of welding, for example fusion welding, laser welding or ultrasonic welding. In another embodiment, it may be secured by means of adhesives (e.g. silicone, epoxy or cyanoacrylate adhesives). Adhesive bonding may be enhanced by prior surface treatment of the layers for example by grit blasting, chemical etching or by treatment with a plasma, corona, laser or UV light. Mechanical surface roughening of the surface may be accomplished using silicon carbide or by sand or mechanical roughening. Suitably, surfaces to be bonded should be first degreased with methylethyl ketone or acetone, roughened and then cleaned again in order to remove debris and grease. Chemical etching of carbon fibre filled PEEK surfaces has been achieved using a composition of K₂Cr₂O₇, H₂O and H₂SO₄, as described in Davies, P., et al., Surface treatment for adhesive bonding on carbon fibre-poly(etheretherkethone) composites. Journal of Materials Science Letters, 1991(10): p. 335-338. Cold gas plasma treatment imparts surface modification by altering the surface chemistry of a polymer and, if carried out long enough, will also have an effect on surface roughening. Typical gases used for the treatment of polymers are air, oxygen, nitrogen, helium, argon and ammonia. Corona treatment utilises a glow discharge similar to plasma treatment but operating in air and at atmospheric pressure. Laser treatment of a material surface is accomplished by exciting either gas or a solid to emit light of a particular wavelength. This energy chemically modifies the surface and promotes surface roughening or ablation. UV-light treatment involves delivering light at wavelengths between 172 nm and 308 nm to alter the surface of a material.

The cover 18 may include a small opening to allow air to be expelled as the cover is placed in position.

In a preferred embodiment, the cover may be secured in position with a silicone (or epoxy) potting compound which fills region 40 and contacts and adheres to the inside surface of the cover 18 when it is secured in position.

Once the PEEK cover is in position, it can provide physical resistance to for example shear and impact forces. Furthermore, the enclosure provides electrical insulation and allows for improved RF telemetry with reduced heating resulting from Eddy current losses.

A preferred polyetherethereketone is PEEK-OPTIMA (Trade Mark) which is a safe, biocompatible and stable polymer. PEEK-OPTIMA® has been extensively tested to ISO 10993 standards and demonstrated no evidence of cytotoxicity, systematic toxicity or irritation. PEEK-OPTIMA® polymer can be repeatedly sterilized using conventional sterilization methods including steam, gamma radiation and ethylene oxide processes without the degradation of its mechanical properties or biocompatibility. PEEK-OPTIMA® polymer is naturally radiolucent and compatible to imaging techniques such as X-ray, MRI and Computer Tomography (CT). The mechanical properties of PEEK-OPTIMA (Table 1) allow for it to meet the physical demands under selected thickness values.

* Physical properties of PEEK-OPTIMA are provided in the table below.

| Property | Method | Value |
|---|---|---|
| Mechanical properties | | |
| Density (g · cm⁻³) | ASTM D792 | 1.3 × 10⁺⁰⁰ |
| Tensile strength (MPa) | ISO 527 Type 1B at | 101 |
| | 50 mm · min⁻¹ | |
| Elastic modulus (GPa) | ASTM D638 TV | 3.5 |
| Elongation at break (%) | ISO 527 Type 1B at | 20-30 |
| | 50 mm · min⁻¹ | |
| Flexural strength (MPa) | ISO 178 | 174 |
| Flexural modulus (GPa) | ISO 178 | 4.2 |
| Izod Notched Impact (kJ · m⁻²) | ASTM D256 | 4.3 |
| Glass transition temperature (° C.) | DSC | 142 |
| Melt temperature (° C.) | DSC | 344 |
| Specific heat capacity | DSC | 2.16 |
| (KJ · Kg⁻¹ · ° C.⁻¹) | | |
| Thermal conductivity coefficient | ASTM C177 | 2.5 × 10⁻⁰¹ |

The impact strength of films of polyetheretherketone which may be used have been tested under ASTM D3763 to confirm that properties are suitable for use in medical devices described. The results of falling weight impact tests for film thickness of 0.1 mm, 0.3 mm and 0.5 mm are shown in FIG. 5.

Furthermore, the electrical insulation properties of the polyetheretherketone, detailed in Table 2, are such that it may advantageously be used in the manner described.

TABLE 2
PEEK electrical properties (23° C., 1 bar, 100 μm film

| Electrical properties | Conditions | PEEK |
|---|---|---|
| Conductivity (S · m⁻¹) | — | 1.50 × 10⁻¹⁵ |
| Dielectric strength (KV · mm⁻¹) | — | 1.98 × 10⁺⁰² |
| Breakdown voltage | | 9.5 |
| (thickness 50 μm, kV) | | |
| Dissipation factor | 1 KH | 2 × 10⁻⁰³ |
| Volume resistivity (Ω · cm) | — | 4.9 × 10⁺¹⁶ |

Although titanium has some advantageous properties it has disadvantageous electromagnetic compatibility (EMC) properties in general and in comparison to polyetheretherketone. As a result, titanium housings have detrimental telemetry characteristics when used for medical devices which are arranged to communicate and/or interact with electrical and/or magnetic fields outside the device. By way of example, as shown in Table 6, a titanium layer of thickness 300 μm would provide a reduction in the electrical field magnitude and energy density of more than 99% for a signal frequency greater than 10 MHz. In contrast, polyetheretherketone has favourable EMC properties, as illustrated in Tables 6 and 7.

TABLE 6
PEEK and Titanium EMC electrical attenuation behaviour
(23° C., 1 bar).

Skin depth (m)

| Conditions | PEEK | Titanium |
|---|---|---|
| 1 MHz | ≈1 × 10¹² | 3.70 × 10⁻⁰⁴ |
| 10 MHz | >>1 | 1.17 × 10⁻⁰⁴ |
| 100 MHz | >>1 | 3.70 × 10⁻⁰⁵ |
| 400 MHz | >>1 | 1.85 × 10⁻⁰⁵ |
| 1000 MHz | >>1 | 1.17 × 10⁻⁰⁵ |

TABLE 7
Amount of power lost by electromagnetic waves traversing through
PEEK and Titanium (signal frequency 400 MHz, Temp 23° C.,
pressure 1 bar).

| | | |
|---|---|---|
| | Reduction in | Reduction in |
| | the electric field | the electric field |
| Thickness | magnitude (%) | energy density (%) |

| (μm) | PEEK | Titanium | PEEK | Titanium |
|---|---|---|---|---|
| 18.5 | ≈0 | 63.2 | ≈0 | 86.5 |
| 100 | ≈0 | >99.3 | ≈0 | >99.7 |

Referring to table 7, considering a titanium layer with a thickness equal to the skin depth, the electric field magnitude is reduced to 36.8% of its incident value and the electric field energy density is attenuated to 13.5% of its initial value.

Compared to current devices which may operate at frequencies of less than 150 KHz due to the thickness of titanium used, arrangements as described herein may allow higher frequencies, for example up to 400 MHz or 800 MHz to be used.

Another electrical property of titanium which is disadvantageous is its influence on attempts to induction charge batteries contained within implantable devices. Table 8 includes calculations on the respective influences of polyetheretherketone and titanium on induction charging, on the basis of implantable battery characteristics displayed in Table 9 and a 15 mm radial enclosure.

TABLE 8
Influence of PEEK and Titanium on implantable battery recharging

| Characteristics | PEEK | Titanium |
|---|---|---|
| Eddy current loss (mW) | ≈0 | 2.0 × 10⁺⁰² |
| Induction heating of casing (° C.) | ≈0 | 4.84 × 10⁺⁰⁰ |
| Battery loss to heating (mW) | 3.2 × 10⁺⁰⁰ | 3.2 × 10⁺⁰⁰ |
| Battery heating (° C.) | 7.8 × 10⁻⁰¹ | 7.8 × 10⁻⁰¹ |

TABLE 9
Implantable battery characteristics.

| Battery property | Value |
|---|---|
| Battery capacity (mAh) | 1.6 × 10⁺⁰² |
| Charge rate of a medical implantable | 8.0 × 10⁺⁰¹ |
| Li battery (mAh) | |
| Peak charging voltage (V) | 4 × 10⁺⁰⁰ |
| Charging power (mW) | 3.2 × 10⁺⁰² |
| Coulombic efficiency (%) | 9.0 × 10⁺⁰¹ |

It will be noted from Table 8 that, whereas titanium exhibits significant Eddy current losses and a heat rise in the casing, polyetheretherketone advantageously has a negligible effect on such properties.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steeps of any method or process so disclosed.

1-21. (canceled)

22. A method of making a medical device, the method comprising:
(a) selecting a film or tubing material which comprises a thermoplastics polymeric material;
(b) selecting a former;
(c) preparing a cover by heat processing the film or tubing on the former so that the film or tubing at least partially adopts the shape of the former; and
(d) securing the cover over an opening in a precursor of the medical device to close the opening.

23. The method according to claim 22, wherein said device has a maximum dimension of at least 5cm and of less than 50 cm, and a maximum diameter of less than 5 cm.

24. The method according to claim 22, wherein said device being elongate and comprising a femoral nail.

25. The method according to claim 22, wherein said device incorporates electronics.

26. The method according to claim 22, wherein said precursor of the device comprises a metal.

27. The method according to claim 26, wherein said metal comprises titanium.

28. The method according to claim 22, wherein the opening in the precursor includes a hollow region of the precursor in which electronics are arranged, wherein the hollow region includes a potting material to secure the electronics within the device.

29. The method according to claim 22, wherein said film or tubing material has a thickness in the range of 25 μm to 1 mm.

30. The method according to claim 29, wherein the thickness is less than 500 μm.

31. The method according to claim 22, wherein said film or tubing comprises a polymeric material which has a moiety of formula of at least one of: 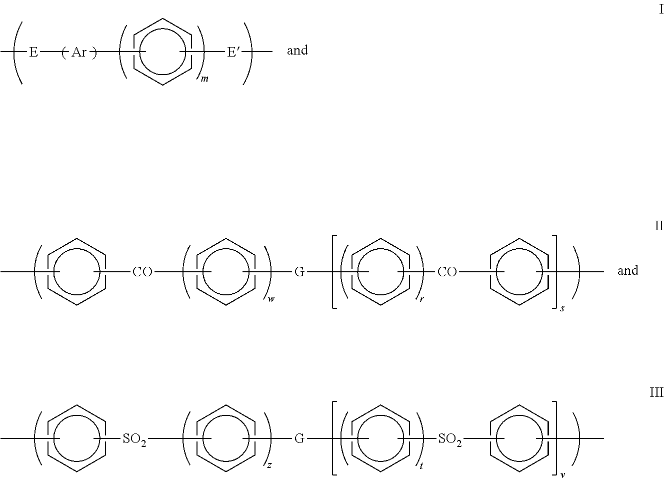
wherein: m,r,s,t,v,w and z independently represent one of zero and a positive integer; E and E′ independently represent one of an oxygen atom, a sulphur atom, and a direct link; G represents one of an oxygen atom, a sulphur atom, a direct link, and a —O-Ph-O— moiety where Ph represents a phenyl group; and Ar is selected from one of the following moieties (i)**, (i) to (iv) which is bonded via one or more of the phenyl moieties to adjacent moieties

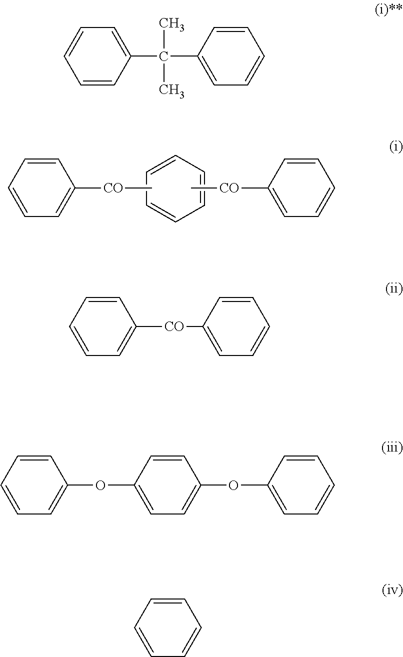

32. The method according to claim 22, wherein said polymeric material comprises a repeat unit of formula (XX) 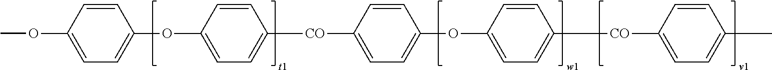
where t1, and w1 independently represent one of 0 and 1, and v1 represents one of 0, 1, and 2.

33. The method according to claim 22, wherein said polymeric material is selected from the group consisting of polyetheretherketone, polyetherketone, polyetherketoneetherketoneketone and polyetherketoneketone.

34. The method according to claim 22, wherein said polymeric material is polyetheretherketone.

35. The method according to claim 22, wherein said film or tubing includes at least 90 wt % of said polymeric material.

36. The method according to claim 22, further comprising contacting the film or tubing with said former and subsequently heating to thermoform the polymeric material on the former.

37. The method according to claim 22, wherein after step (c), there is no further deformation of the cover.

38. The method according to claim 22, further comprising prior to securing the cover over the opening in the precursor of the medical device in order to close the opening, introducing a potting compound to the opening and contacting and adhering the cover to the potting compound.

39. The method according to claim 22, wherein securing the cover over the opening in the precursor of the medical device to close the opening further comprises positioning the cover over the opening to completely close the cover and secured the cover in position.

40. A medical device comprising a precursor of a medical device in which an opening is defined and a cover secured over the opening, said cover comprising a thermoplastics polymeric material.

41. A method of treating a human body using a medical device, the medical device made in a process comprising:
(a) selecting a film or tubing material which comprises a thermoplastics polymeric material;
(b) selecting a former;
(c) preparing a cover by heat processing the film or tubing on the former so that the film or tubing at least partially adopts the shape of the former; and
(d) securing the cover over an opening in a precursor of the medical device to close the opening.
